(19) United States
(12) Patent Application Publication
(10) Pub. No.: US 2025/0160744 A1
(43) Pub. Date: May 22, 2025

(54) FUNCTIONAL TREMOR RETRAINMENT DEVICE

(71) Applicant: UNIVERSITY OF VIRGINIA PATENT FOUNDATION, Charlottesville, VA (US)

(72) Inventor: Jordan F. Garris, Charlottesville, VA (US)

(21) Appl. No.: 18/951,443

(22) Filed: Nov. 18, 2024

Related U.S. Application Data
(60) Provisional application No. 63/599,782, filed on Nov. 16, 2023.

(51) Int. Cl. A61B 5/00; A61B 5/11; A63B 24/00; A63B 71/06

(52) U.S. Cl. CPC A61B 5/486; A61B 5/1101; A61B 5/4836; A63B 24/0075; A63B 71/0622; A61B 2505/09; A61B 2562/0219; A63B 2071/0625

(57) ABSTRACT

Various technological aspects of functional tremor retrainment are described. For example, a mobile device may measure a baseline tremor frequency, determine a cue frequency based on the baseline tremor frequency, provide motion cues at the cue frequency, measure a motion frequency while providing the motion cues, and provide a feedback signal indicative of a synchrony of the motion frequency to the cue frequency.

### CROSS-REFERENCE TO RELATED APPLICATIONS

This application claims the benefit of the priority of U.S. Provisional Patent Application No. 63/599,782, filed on Nov. 16, 2023, and titled, “Tremor Retrainer Smartphone Application For The Treatment Of Functional Tremor,” the contents of which are hereby incorporated in their entirety.

### STATEMENT REGARDING FEDERALLY SPONSORED RESEARCH OR DEVELOPMENT

N/A

### TECHNICAL FIELD

Various embodiments and implementations described herein relate generally to systems and methods for the treatment of functional movement disorder, such as functional tremor. More specifically, embodiments and implementations hereof may involve functional tremor therapy and/or retrainment.

### BACKGROUND

Functional movement disorders (FMDs) are a common and costly source of health care utilization and disability. Patients with FMD comprise 3-23% of presentations for neurologic symptoms and often experience significant disability and poor prognosis for recovery. The incidence of FMDs has increased during the COVID-19 pandemic with a 75% increase in diagnoses from 2019 to 2020. Functional tremor—the most common FMD—causes persistent disability in 44-90% of cases with symptoms that usually continue for years. Common disabilities resulting from functional tremor include difficulty with activities of daily living and inability to work. Patients with FMDs report similar levels of disability compared to those with neurodegenerative and other neurologic disorders.

Early diagnosis and treatment improve prognosis in functional neurological disorders, but treatment is often delayed. Because FMD is a subset of functional neurologic disorder, much data pertains to functional neurologic disorder as a whole. The most promising line of treatment for functional neurologic disorders—mainly studied in open-label designs—centers on cognitive-behavioral therapy and intensive rehabilitation with physiotherapy.

However, therapies to treat FMD are resource intensive, and access to FMD-trained psychologists and inpatient rehabilitation centers is limited. A recent panel of young adults with functional neurologic disorder noted frustration not only with delay in diagnosis, but also with dismissal by the medical system after receiving a diagnosis with no treatment provided. The same panel also noted that treatment options were even more constrained for adolescents since most programs available for functional neurologic disorder were focused on adults. The perception of abandonment can impair recovery; a patient in the panel noted that receiving a diagnosis even within 1 week of symptom onset “didn't mean anything” because no treatment was offered. FMD patients also face the challenge of treatment delays, waiting months for any psychotherapy, particularly for therapy from a psychologist with experience in FMD.

Pediatric patients are also highly impacted by FMD. Because FMD is a subset of functional neurologic disorder, much data pertains to functional neurologic disorder as a whole. A recent prospective study demonstrated an annual incidence rate of functional neurologic disorder of 18.3 per 100,000 children aged 5-15, 22 nine times the global incidence rate of multiple sclerosis. While several earlier studies suggest that pediatric patients are more likely to improve following diagnosis and treatment of functional neurologic disorder than adults, only half of patients had a good outcome at median follow-up of 15 months, with 80% reporting ongoing symptoms. Pediatric patients can experience continued functional symptoms years later: 23% of patients with pediatric functional neurologic disorder have sufficient burden of functional symptoms to be included in medical records in adulthood. Accordingly, including pediatric patients in early treatment trials in FMD is important because treating patients with pediatric onset FMD could provide a window of opportunity to avoid the risks of long-lasting disability in adulthood.

Because FMD, including functional tremor, is a class of neurological condition, it differs significantly in several important was from other types of tremors or involuntary body movements (such as those caused by Parkinson's disease, for example). Because of these differences, both the presentation of, and treatment of, FMD does not equate to the same types of symptoms and treatment that are used for other tremor-like conditions.

First, FMD is generally a neurological condition related to how the brain controls movement. It is not necessarily caused by structural damage or conditions of the nervous system, like structural brain/nerve damage, neurodegeneration, etc. Instead, it can be influenced by psychological factors like stress, anxiety, or trauma. In contrast, other involuntary movement disorders like Parkinson's tremor, essential tremor, and dystonia are associated with underlying structural or biochemical changes in the nervous system.

Second, clinical presentation of FMD differs from other conditions. FMD can exhibit a higher degree of variability (such as starting/stopping, or changing frequency based on the patient's attention or distraction); can be inconsistent in terms of its patterns or combinations of movements; and can exhibit sudden onset and progression. Other tremors and involuntary movement disorders tend to be more consistent and predictable, and while they may worsen with certain types of stress, they usually do not change based on the patient's degree of distraction. Instead, they can be influenced by physical action (e.g., postural adjustments, purposeful physical activity, etc.).

As such, typical treatment approaches for involuntary movement conditions other than FMD are not directed to the underlying causes and presentation of FMD, and are not necessarily appropriate to be applied to patients with FMD such as functional tremor. In fact, there is a significant lack of resources and properly trained therapists for FMD-like conditions, which even further leads to worsening of symptoms due to feelings of hopelessness and inability to attain treatment.

Therefore, a need exists for a system and method that can be made available to FMD patients that provides easily accessible treatment regimes that are tailored to the underlying causes and symptomatic presentation of these patients' conditions.

### BRIEF DESCRIPTION OF THE DRAWINGS

FIGS. 1A-1D illustrate an example mobile device executing a functional tremor retrainment mobile application.

FIG. 2 illustrates example paired mobile devices executing functional tremor retrainment mobile applications.

FIG. 3 illustrates an example method of operation of a mobile device providing functional tremor retrainment.

FIG. 4 illustrates a block diagram of an example 400 upon which one or more embodiments can be implemented.

### DETAILED DESCRIPTION

Although example embodiments of the present disclosure are explained in some instances in detail herein, it is to be understood that other embodiments are contemplated. Accordingly, it is not intended that the present disclosure be limited in its scope to the details of construction and arrangement of components set forth in the following description or illustrated in the drawings. The present disclosure is capable of other embodiments and of being practiced or carried out in various ways.

It should be appreciated that any element, part, section, subsection, or component described with reference to any specific embodiment above may be incorporated with, integrated into, or otherwise adapted for use with any other embodiment described herein unless specifically noted otherwise or if it should render the embodiment device nonfunctional. Likewise, any step described with reference to a particular method or process may be integrated, incorporated, or otherwise combined with other methods or processes described herein unless specifically stated otherwise or if it should render the embodiment method nonfunctional. Furthermore, multiple embodiment devices or embodiment methods may be combined, incorporated, or otherwise integrated into one another to construct or develop further embodiments described herein.

The diagnosis of functional tremor is made by demonstrating tremor entrainment, an exam feature in which an externally cued rhythmic movement leads the tremor to “entrain”, that is, adopt the cued frequency. Demonstration of this “positive sign” of FMD is a major criterion for the diagnosis of functional tremor and is common to both pediatric and adult onset functional tremor. While critical for diagnosis, entrainment may be used as a therapeutic strategy, repurposed as “retrainment.” Such tremor retrainment could serve as a biofeedback strategy, allowing the patient to recognize that redirection of attention to a task, rather than their symptoms, can suppress or change the functional manifestation. This strategy targets two key elements of pediatric and adult functional movement disorders: impaired self-agency and impaired attention. Principles of treatment with tremor retrainment overlap with the motor reprogramming strategy used in several inpatient rehabilitation protocols for functional movement disorders which have been associated with re-organization of connectivity between emotional processing and motor planning networks on functional magnetic resonance imaging (fMRI).

Physician led tremor retrainment therapy is very resource intensive, limiting its usefulness and accessibility to patients. Additionally, pediatric patients have historically been excluded from clinical trials in functional neurologic disorder and face even greater barriers in accessing treatment as a result.

Aspects of the disclosed technology address such challenges by providing technology that may be implemented via a patient's mobile device to provide retrainment therapy. For instance, a smartphone application which would be immediately accessible to most patients at the time of diagnosis. The described technology may be useable by adult and pediatric patients. Accordingly, the described technology may provide reduction and/or resolution of functional tremor symptoms if treatment is effective and availability of treatment in a home-based setting.

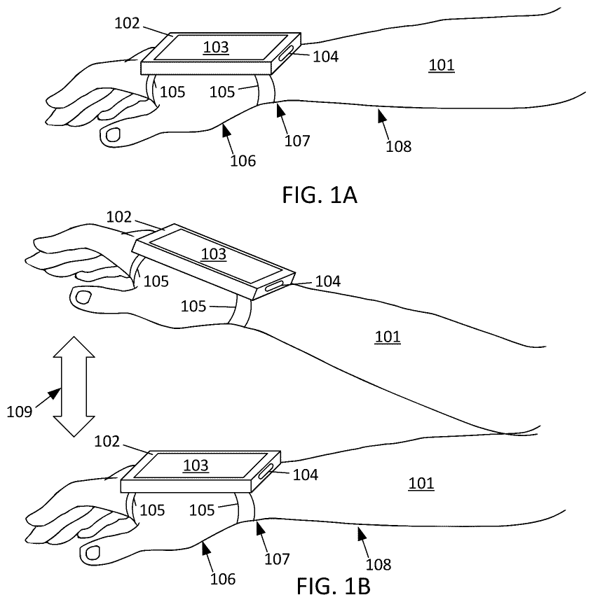

FIGS. 1A-1D illustrate examples of a user 101 performing functional tremor retrainment in accordance with a retrainment process performed by a mobile device 102. For example, mobile device 102 may comprise a smartphone, smartwatch, fitness tracker, or other wearable device. In the illustrated example, mobile device 102 may execute a mobile application providing tremor retrainment therapy (“retrainment app”).

In some examples, mobile device 102 may be mounted to a distal portion of a user's arm 101. For example, mobile device 102 may be mounted to arm 101 via a strap 105. In some cases, mobile device 102 may be mounted to arm 101 such that mobile device 102 visible to the user when their arm 101 is in a pronated position (e.g., as illustrated). In other cases, mobile device 102 may be mounted to arm 101 to be viewable in other positions, such as a supinated position, a neutral position, etc. In some examples, mobile device 102 may execute the retrainment app to provide information to the user regarding an instructed arm position. For instance, the retrainment app may display an example image/illustration of a properly worn mobile device.

In some examples, mobile device 102 may be mounted to arm 101 at the user's wrist 107. For example, mobile device 102 may be located such that it partially overlaps with the user's hand 106 (e.g., dorsal surface) and the user's forearm 108. As another example, mobile device 102 may be located so that it overlaps with the user's wrist 107 and forearm 108, only overlaps the user's forearm 208, overlaps the user's hand 106 and wrist 107, only overlaps the user's hand 106, or any other suitable arrangement. For instance, strap 105 may comprise a sleeve retaining mobile device 102, a plurality of straps at various locations on the user's arm, or other suitable wearable mobile-device retaining device. As another example, mobile device 102 may comprise a smart watch and strap 105 may comprise a watchband.

In some examples, mobile device 102 may execute the retrainment app to determine a user's baseline movement frequency. In some examples, the baseline movement frequency may be measured at the initiation of each retrainment session, such that it represents an initial motion frequency upon commencement of the retrainment session. For example, variability in tremor frequency may be associated with functional tremor. Acquisition of the baseline tremor frequency at the beginning of each retrainment session may accommodate changes in the user's baseline tremor frequency between sessions. In some examples, the baseline movement frequency may be acquired over a period of time, as the average or median movement frequency.

The baseline tremor frequency may be measured via several suitable methods. For instance, where the user exhibits functional movement disorder as an arm, hand, or wrist movement, a user may be directed to extend their arm 101 horizontally in front of them and to maintain their arm 101 in that position while their baseline tremor frequency is measured. For instance, mobile device 102 may comprise an accelerometer, gyroscope, inertial measurement unit (IMU), etc. that can record motion-related data. For example, mobile device 102 may record motion data as the user extends their arm for a set time, until the retrainment app is able to determine a baseline tremor frequency, etc. As an example, mobile device 102 may execute the retrainment app to display a signal to the user to begin or to end maintaining the baseline tremor acquisition position on display 103. In other situations, where the patient exhibits functional movement disorder at another body part (e.g., another limb such as a leg/ankle, etc.), the patient may be directed to move or extend the associated body part to exhibit the functional movement disorder's current frequency.

In some examples, the baseline tremor frequency may correspond to a lowest/fundamental frequency of a user's functional tremor. For example, the baseline tremor frequency may be obtained via a signal analysis technique such as Fourier analysis (e.g., a Discrete/Fast Fourier Transform (DFT/FFT)), low pass filtering, etc. In some examples, the baseline tremor frequency may be determined based on an expected range of frequencies. For instance, bandpass filtering may be used to isolate a certain frequency range of the motion data, such as between about 5-12 Hz (4-13 Hz) to capture a typical functional tremor that may be between 6-11 Hz. After filtering, further signal analysis may be performed on the filtered data to determine the baseline tremor frequency.

In some examples, the retrainment app may comprise a minimum baseline tremor frequency or a maximum tremor frequency. For example, the retrainment app may set the tremor frequency to the minimum frequency responsive to lack of detection of a tremor, detection of a tremor below the minimum frequency, etc. Continuing, the retrainment app may set the tremor frequency to the maximum frequency responsive to detecting a tremor at a frequency above the maximum. For instance, a user may display a tremor having multiple frequency components, including a main component at a frequency above the maximum frequency threshold. For example, the minimum and maximum frequency may be established according to ranges at which functional tremor presents.

In some examples, mobile device 102 may execute the retrainment app to guide a user through a tremor retrainment session. For example, a user may be directed to raise and lower 109 their hand/wrist/forearm at a determined retrainment cue frequency. For example, mobile device 102 may provide motion cues to the user at the cue frequency. For instance, mobile device 102 may provide pulsed signals at the cue frequency, such as, for example, pulsed audio tones via a speaker 104, pulsed visual signals (e.g., flashes, animations, etc.) via display 103, pulsed haptic signals via a transducer/vibration motor (not pictured), etc. As an aid in explanation, further examples may be described primarily with respect to audio cues; any described example may utilize any suitable technology to provide motion cues to the user at the cue frequency.

In some examples, the cue frequency may be based on the baseline tremor frequency. For example, the cue frequency may be different from the baseline tremor frequency. For instance, the cue frequency may be a fixed number of Hz above or below the baseline tremor frequency (e.g., between about 1-7 Hz). As another example, the cue frequency may be a portion/percentage of the baseline tremor frequency (between ⅓-⅔ of the baseline tremor frequency). In various examples, the cue frequency may be any frequency different from the baseline frequency. For example, 1-99%, 5-80%, 10-70%, 15-65%, etc.

In some cases, certain frequencies related to the baseline tremor frequency may be avoided. For example, humans may be more easily able to synchronize to a rhythm (e.g., “feel” a beat) that is a multiple of a power of 2 with respect to the base frequency (e.g., multiples of ½, ¼, ⅛). For example, humans may tend to more easily follow a rhythm of a frequency that is ½ the baseline frequency compared to other fractions (e.g., if listening to a baseline 12 Hz tone pulses, then a human may more easily synchronize with a 6 Hz rhythm (½ the baseline) than an 8 Hz rhythm (⅔) of the rhythm. As indicated above, functional tremor may be associated with a loss of a sense of self-agency. A user following a cue that is ½ the user's tremor frequency may provide have less of a feeling of self-agency compared to a cue that is ⅓ or ⅔ of the tremor frequency. Of course, this merely explains a theory of action, and implementations are not limited to avoiding such frequencies.

In some examples, mobile device 102 may execute the retrainment app to provide different cue frequencies over the course of a treatment session. For example, mobile device 102 may provide a first cue frequency for a first portion of a session and a second cue frequency for a second portion of the session. As an example, a treatment session may include two phases, where a first phase has a cue frequency greater than 50% of baseline tremor frequency and a second phase has a cue frequency less than 50% of baseline tremor frequency. For example, a cue frequency might be about ⅔ of the baseline tremor frequency during a first phase and about ⅓ of the baseline tremor frequency during a second phase. In various examples, different phases may have any suitable lengths. For instance, the first phase may be longer or shorter than the second phase or both phases may be substantially equal in length. As another example, a treatment session may include more than two phases, such as 3, 4, 5, etc. phases.

As a further example, cue frequency may vary in other manners over the course of a session. For example, mobile device 102 may execute the retrainment app to gradually change the frequency, stepwise change the frequency, etc. In some examples, the cue frequency may be responsive to inputs or measurements made during the session. For instance, the cue frequency may transition to a second rate responsive to the user meeting the first cue rate for a sufficient period of time.

In some examples, the cue frequency may be based on a treatment session. For instance, during an initial phase of retrainment therapy, the cue frequency may be higher than during a later phase of retrainment therapy. Generally, earlier treatment phases might have cue frequencies than later cue frequencies (e.g., a user initially retrains at a frequency closer to their tremor frequency and later retrains at a frequency farther from their tremor frequency.

As another example, a course of therapy may include a first or “induction” session followed by second or “maintenance” sessions. In this example, the induction session may be longer than the maintenance sessions. For instance, an induction session may be between about 1 and about 2 hours (e.g., a 60 min induction session) and maintenance sessions may be between about 10 mins and about 45 mins (e.g., 15 min, 20 min, 30 min maintenance sessions). As a further example, sessions may vary in time throughout the course of treatment. For instance, maintenance sessions may decrease in duration over a period of time, be randomized, etc. In some examples, a course of therapy may comprise a set total number of hours of retrainment, such as a total of between about 2 and about 6 hours (e.g., spread out over a number of days). As another example, a course of therapy may take place over a number of weeks, months, etc. As a particular example, a 60 minute induction session may comprise a 30 min phase at ⅓ the baseline tremor frequency and a second 30 min phase at ⅔ the baseline tremor frequency. Similarly, a particular 30 or 15 min maintenance session might comprise a 15 or 7.5 min phase at ⅓ the baseline tremor frequency and a 15 or 7.5 min phase at ⅔ the baseline tremor frequency. Of course, this is simply an illustrative example; treatment sessions may be performed according to various parameters as discussed herein.

In such examples, the mobile device 102 may receive an input indicative of a treatment session. For example, as illustrated in FIG. 1C, a retrainment app executed by mobile device 102 may display a user interface on display 103 to enable a user to select their training session. For instance, as illustrated, a user may select a first session 110 or a second session 111. In further examples, additional sessions may be available. As another example, mobile device 102 may receive a date/time input (e.g., from the user, internal clock, time server, etc.) as an indicator of the treatment session. For instance, during a first period (e.g., a first week after beginning therapy), the retrainment app may perform sessions of a first type and during a second period of time (e.g., 1-2 months following the second period of time), the retrainment app may perform sessions of a second type. Of course, implementations are not limited to any particular treatment plan, session timing, phase length, etc.; the described technology may support any retrainment therapy parameters. Thus, the user interface on mobile device 102 may comprise a displayed graphical user interface allowing the wearer to view options, select settings and actions, view cue frequency prompts, view training session frequency (non) alignment to the cue frequency, etc. In some examples, the user interface may also allow the user to input information (whether freeform, by answering questions, adjusting rating scales, etc.) regarding their perception of the training session (e.g., its perceived difficulty, whether symptoms were perceived to have improved since the prior session, whether symptoms seemed to improve after the current session, how the session affected their mental state, etc.) In further examples, the user interface may also present the user with an option to provide non-tremor-frequency, non-training information such as descriptions of their overall stress, anxiety, manifestations of trauma, or other factors that can impact functional movement disorders. For example, in association with a given training session, a user may have the ability to enter information relating to their state of anxiety; current feelings of stress; depressive or trauma-related thoughts, emotional struggles, sleep, hyperawareness of symptoms, fear of certain activities, performance pressure, and/or injury recovery, through standardized responses to a series of questions presented to the user. This information can then be stored either on the mobile device 102 itself, or may be sent to the user's healthcare provider to be stored in their electronic medical records along with a training session summary.

As discussed above, mobile device 102 may execute a retainment app to provide motion cues to a user at the cue frequency. For example, mobile device 102 may emit pulsed tones or other audio cues via a speaker 104. A user may be directed to raise and lower 109 their arm in a rhythm matching the motion cues. For example, a user may be directed to extend their arm in front of them and raise and lower their arm via extension/flexion of their wrist, elbow, shoulder, or combinations thereof. Additionally, in some cases, a user may be directed to assume a particular treatment position while following the cues. For instance, a user may be directed to stand or sit and extend their arm horizontally forward or to the side. As another example, a user may be directed to choose their positioning, such as to find a comfortable position with their arm extended horizontally in some direction.

In some examples, a user may be directed to perform motions 109 in a particular manner, through visual or auditory notifications from the user interface. For instance, a user may be directed to raise and lower their arm parallel to the sagittal plane without transverse motion (e.g., “straight up and down”). In other examples, a user may be directed to perform other rhythmic motions 109, such as moving their hand side-to-side. As another example, a user may be directed to perform the rhythmic motions 109 in a manner of their choosing. In some implementations, mobile device 102 may execute the retrainment app to monitor the user's compliance with a directed motion. For instance, accelerometer data or other IMU data may be used to detect a user deviating from the directed motion by more than a threshold amount. In such examples, mobile device 102 may provide a warning or other indicator to the user (e.g., graphic displayed on user interface via screen 103).

In some examples, mobile device 102 may provide a feedback signal indicative of a synchrony of the motion frequency to the cue frequency. For example, providing the feedback signal while a session is ongoing may assist a user feeling a sense of agency over their movements. For instance, the feedback signal may be a visual feedback signal, audio feedback signal, haptic feedback signal, etc. For example, FIG. 1D illustrates an example where the feedback signal comprises a visual feedback signal providing a visual indication of a difference between the motion frequency and the cue frequency. As illustrated, the visual feedback signal may comprise a gauge graphic 112. In various examples, the feedback signal may provide feedback to the user regarding how synchronized their motions 109 are to the cue frequency. For instance, mobile device 102 may obtain accelerometer or other IMU data during movements 109 to determine a frequency at which the user is moving 109. Mobile device 102 may determine a difference between the motion frequency and the cue frequency and provide a feedback signal indicative of this difference. For example, as illustrated, a graphic 112 comprising an indicator 113 (e.g., a pointer, arrow, or other “gauge needle”) further comprises a region 115 indicative of a user moving 109 within a threshold range of the motion cues. In this example, graphic 112 further comprises regions 114, 116 indicative of a user moving too slowly or too quickly, respectively. In such examples, the threshold of acceptable synchronicity may be determined in any suitable manner. For example, the threshold may be between about 0.1 Hz and about 2 Hz lower/higher than the cue frequency. For instance, the threshold may be 0.5 Hz+/− of the cue frequency (e.g., a threshold range of 1 Hz). In some examples, the threshold may be symmetric about the cue frequency, while in others the threshold may be asymmetric (e.g., the threshold indicative of moving too fast may be greater or lower than the threshold of moving too slow). In some examples, the feedback signals may be based on various other factors, such as treatment session, user progress, etc. For instance, during a first or earlier treatment phase, the threshold for acceptable synchronicity might be higher than during a later treatment phase.

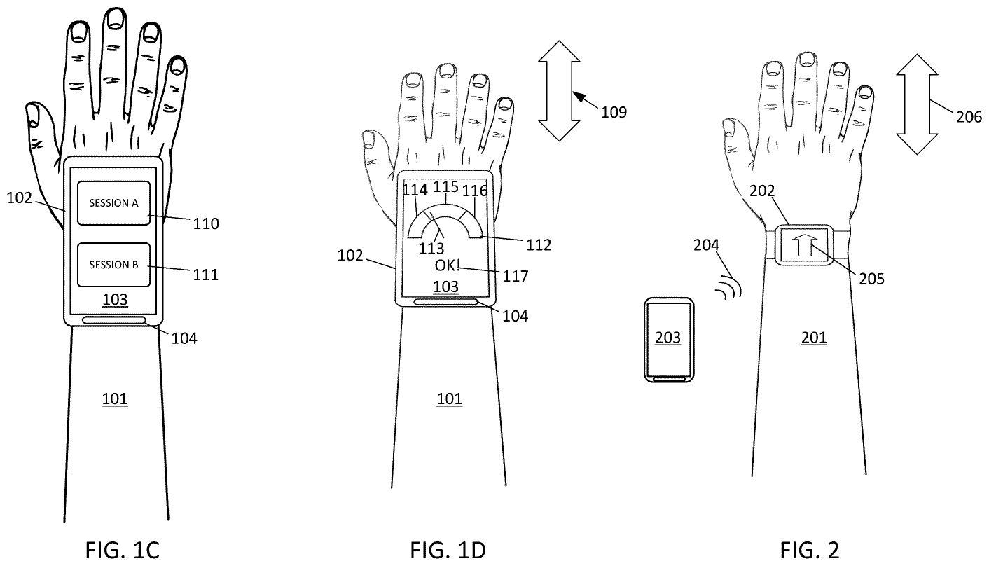

In some examples, the feedback signal may be indicative of a degree to which the user's motion frequency 109 differs from the cue frequency. For instance, in the illustrated example, the angular distance of indicator 113 from the center of region 115 may be indicative of the degree that the user's motion 109 differs from the cue frequency. In further examples, other or additional feedback signals 117 may be included, such as a first signal indicating the state of synchronicity (e.g., text displaying “Too Fast!”, “Too Slow!”, “Good!”, etc.) and a second signal indicating the degree of synchronicity. For instance, such text might be displayed underneath gauge 112. In some examples, such text 117 may be descriptive of a position within a region 114, 115, 116. For instance, with indicator in region 115, text 117 may be selected from a set encouraging a user to maintain a center-indicator 113 (e.g., text such as “Ok”, “Good”, “Great”, “Good”, “Ok” may be displayed in a sequence where indicator 113 traverses region 115). As another example, FIG. 2 (discussed further below) illustrates an example mobile device 202 displaying an icon 205 indicative of the synchrony between the user's motion frequency 206 and the cue frequency (e.g., an up arrow indicative that the user is moving too slowly and should be moving more quickly).

Further, in some examples certain settings defining training session attributes such as the cue frequency, the session-to-session variance of cue frequency, particulars of the indication of alignment or non-alignment of the user's training session motion frequency versus the cue frequency, can be set and/or adjusted to be personalized to the user. For example, a healthcare provider that directs the patient to use the application may provide a set of account credentials to the user which are associated with an account that has a set of pre-loaded settings and a regimen. The cue frequency, range of “Good” alignment to the cue frequency, tone of textual or audio feedback, etc. can be adjusted based upon the patient's specific diagnosis and potential underlying causes of the functional movement disorder. For example, a patient that has experienced trauma and performance anxiety may be prescribed a regimen that includes a selection of feedback signal format (from, e.g., several options presented via a dashboard, as described below) that will be more helpful to the patient, such as one that contains more substantial positive reinforcement and a wider range of acceptable “alignment” of the patient's actual motion during retrainment sessions with the cue frequency. Settings such as these may be modified by the healthcare provider on a periodic basis, and/or the healthcare provider may predefine a rate of increase in “difficulty” or advancement in the sessions.

In some examples, mobile device 102 may execute the retrainment app to provide the feedback signal continuously during a treatment session. As indicated above, the feedback signal may be an aspect of the retrainment therapy. For example, it may provide biofeedback for the user to assist a sense of agency and/or redirect attention from the functional tremor. In some examples, if the user is not able to accurately recreate the cue frequency, the software application running via the mobile device 102 may dynamically adjust the session settings, such as increasing or decreasing the cue frequency, shortening or lengthening the session duration, or inviting the user to self-adjust settings to create a better experience.

FIG. 2 illustrates an example implementation comprising a user 201 performing functional tremor retrainment in accordance with a retrainment processes executed by multiple mobile devices 203, 202. As illustrated, in some examples, various aspects of a retrainment app may be distributed amongst multiple mobile devices. For instance, mobile devices 203, 202 may comprise a smartphone 203 paired 204 with a smartwatch 202 (e.g., via a Bluetooth, WiFi, wireless body-area-network (BAN), etc.). Accordingly, in some implementations, a smartwatch may execute a retrainment app independently as discussed above, in further implementations a smartwatch may execute a retrainment app as instructed by a paired mobile device 203.

In this example, mobile devices 203, 202 may each execute a retrainment app to cause the devices to operate as discussed above. In various implementations, retrainment app code may cause the different mobile devices 203, 202 to perform different functions. For example, a first mobile device 203 may comprise a processor and non-transitory computer readable medium storing instruction to cause the processor to execute operations via second mobile device 202.

In some examples, first mobile device 203 may measure a baseline tremor frequency via second mobile device 202. For example, first mobile device 203 may transmit an instruction to second mobile device 202 to begin a retrainment session. As another example, first mobile device 203 may instruct second mobile device 202 to record accelerometer data. In some examples, second mobile device 202 may process the accelerometer data to measure the baseline tremor frequency as instructed by first mobile device 203. In other examples, second mobile device 202 may transmit accelerometer data to first mobile device 203 such that first mobile device 203 processes the accelerometer data to measure the baseline tremor frequency.

In some examples, first mobile device 203 may determine a cue frequency based on the baseline tremor frequency. For example, first mobile device 203 may instruct second mobile device 202 to determine the cue frequency. As another example, first mobile device 203 may determine the cue frequency and transmit it to second mobile device 202.

In some examples, first mobile device 203 may provide motion cues at the cue frequency. For instance, first mobile device 203 may provide auditory cues at the cue frequency using a speaker. For example, first mobile device 203 may emit the auditory cues via its speaker. As another example, second mobile device 202 may emit the auditory cues via its speaker as instructed by first mobile device 203.

In some examples, first mobile device 203 may measure a motion frequency of an arm 201 via second mobile device 202. For instance, first mobile device 203 may instruct second mobile device 202 to process its accelerometer data to measure the motion. As another example, second mobile device 202 may transmit recorded accelerometer data to first mobile device 203. In this example, first mobile device 203 may processes the received accelerometer data to determine the motion frequency.

In some examples, first mobile device 203 may provide a feedback signal indicative of a synchrony of the motion frequency. For example, first mobile device 203 may provide a visual feedback signal via its display (e.g., as discussed with respect to FIG. 1D). As another example, first mobile device 203 may instruction second mobile device 202 to provide a feedback signal 205.

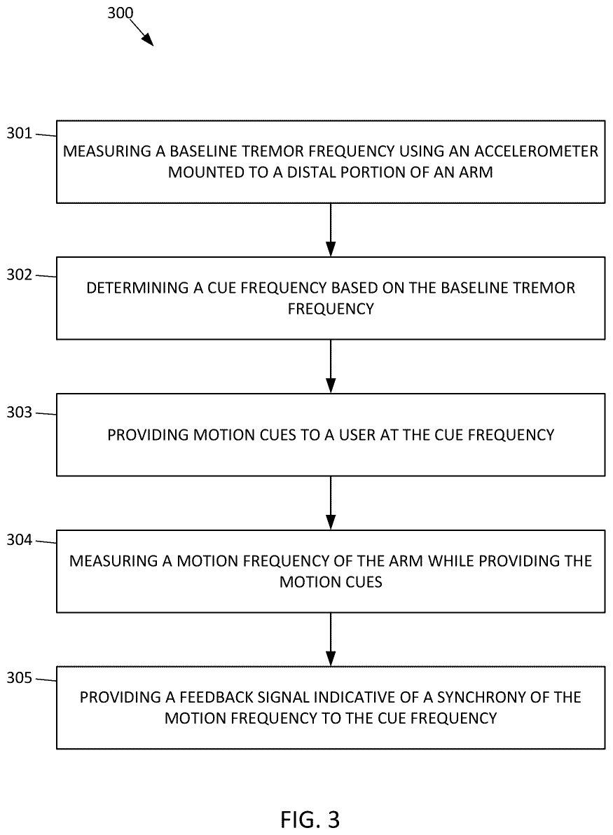

FIG. 3 illustrates an example method 300 of providing functional tremor retraining via a mobile device. For example, method 300 may illustrate an example method of operation of a mobile device such as mobile device 103 of FIGS. 1A-D, mobile devices 202, 203 of FIG. 2, or any other mobile device described herein.

Example method 300 may include process 301, which may include measuring a baseline tremor frequency of a user. For instance, the baseline tremor frequency may comprise an average frequency, principal frequency, etc. of a functional tremor. For example, process 301 may include a mobile device measuring a baseline tremor frequency using an accelerometer mounted to a distal portion of an arm, such as described with respect to FIGS. 1A-1D, 2. For example, peak to peak values, or a cadence of similar changes in directional force, of a raw accelerometer signal may be used to determine a frequency. Alternatively, in some embodiments, the electrical output signal of a device (such as device 102) corresponding to a patient's hand/arm movement may be filtered, smoothed, and/or converted to frequency domain to determine a tremor frequency.

Example method 300 may further include process 302, which may include the mobile device determining a cue frequency based on the baseline tremor frequency. For example, the cue frequency may be determined as described above. In further examples, process 302 may further include the mobile device determining multiple cue frequencies. For instance, process 302 may include the mobile device determining a first cue frequency and a second cue frequency based on the baseline tremor frequency for a session comprising multiple movement cue phases. In some embodiments, method 300 may also comprise a wireless connection to permit a healthcare provider (e.g., physical therapist, clinician, psychologist, etc.) to select cue frequencies for a patient, or to periodically supply settings or preferences from which the mobile device may determine an appropriate cue frequency, such as altering cue frequency according to age and physical health factors and/or mental stress factors (such as impact of an inability to keep up with the cue frequency).

Example method 300 may further include process 303, which may include the mobile device providing motion cues to a user at the cue frequency. For example, process 303 may include providing visual, audio, or haptic cues as described above. For instance, process 303 may comprise providing pulsed audio motion cues to the user via a speaker. In other examples, music having a given beat may be played (e.g., through interface with streaming music accounts), a modification of music having a given beat (in which a drum, baseline, etc. is emphasized, or an additional beat noise is superimposed), a light may flash, and/or a vibration may be implemented, in any combinations thereof. In some examples, process 303 may comprise providing motion cues at different frequencies during a treatment session. For example, process 303 may include the mobile device providing motion cues to the user at the first cue frequency for a first portion of a session; and the mobile device providing motion cues to the user at the second cue frequency for a second portion of the session. In some examples, process 303 may comprise providing motion cues during the entirety of a training session, such as for 1-2 hours, 10-60 mins, etc., as discussed above.

Example method 300 may further include process 304, which may include measuring a motion frequency of a user (e.g., a user's arm) while providing the motion cues. For example, process 304 may comprise receiving accelerometer data and processing the accelerometer data to determine the motion frequency (e.g., via principal frequency analysis, DFT analysis, FFT analysis, etc.).

Example method 300 may further include process 305, which may include providing a feedback signal indicative of a synchrony of the motion frequency to the cue frequency. For example, process 305 may include providing a positive feedback signal when the motion frequency is within a range of the cue frequency. For example, the positive feedback signal may be indicative of the user successfully synchronizing their motions to the motion cues (e.g., retraining). In some examples, process 305 may further include providing a negative feedback signal when the motion frequency is outside the range of the cue frequency. For example, the negative feedback signal may be indicative of the user unsuccessfully synchronizing their motions to the motion cues. For instance, the negative feedback signal may be indicative of the motion frequency exceeding a range threshold or falling below a range threshold. In some examples, process 305 may comprise providing a continuous feedback signal during the session. For example, a user may obtain biofeedback via the feedback signal to assist their attention and/or sense of agency. As described above, the feedback signal may be provided in various manners, such as, for example via visual feedback, auditory feedback, haptic feedback, etc. As a particular example, the feedback signal may comprise a gauge graphic displayed on a mobile device display. In further examples, the visual feedback signal may include a text graphic.

In another aspect, the present disclosure contemplates a dashboard usable by a healthcare provider to enable access to and settings, regimens, and other aspects of using software that implements the processes described herein, such as process 300. Such a dashboard may be integrated within a provider-side electronic medical record (EMR) implementation, or may be a standalone dashboard. In some examples, the dashboard may initially be populated (whether by the healthcare provider or via data extraction from the patient's EMR) with information regarding the patient that can be used to inform how the application should operate. For example, a healthcare provider may initiate an account for the patient, with an initial regimen selection (once per day, every other day, multiple times per day, once per week, or other periodicities as described herein), modality options for deployment of the application (e.g., mobile phone strapped to wrist, smartwatch, or an application specific device to be attached to an arm, leg, or other affected body part), settings for format of how feedback should be provided to the patient during a training session (e.g., visual, auditory, range of synchrony considered “Good”, tone of textual or auditory feedback, etc.), and/or other questions or information that should be solicited from the patient in association with the training session (e.g., questions about the patient's state, as described above).

Because the retrainment sessions are always performed via the software application, the dashboard may also indicate to the healthcare provider the extent to which the patient has adhered to each session, their degree of alignment during the session (including where multiple cue frequencies were used), and their responses to patient state questions). The dashboard may thus provide a graphical representation of the user's progress, and can associate patient state factors with changes in performance. Given this information, the healthcare provider may be able to make further recommendations for treatment or other interventions.

Additionally, the dashboard allows the healthcare provider to offer an immediate avenue for the patient to begin treatment, and to reduce difficulty/barriers to seeking treatment. As described above, current healthcare options for patients suffering from FMD are limited, under-resourced, and difficult to obtain in many cases. Due to the psychological nature of many cases of FMD, feelings of hopelessness and frustration can be quite detrimental to recovery. Thus, by allowing the patient to begin right away, via an account that has already been set up in way that is tailored to the type of treatment and modalities they need, such difficulties can be substantially ameliorated.

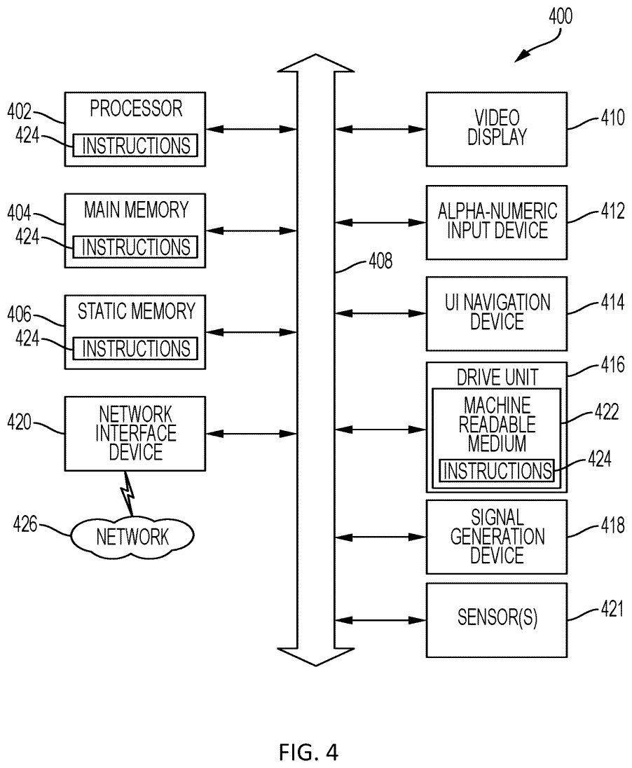

Referring to FIG. 4, an aspect of an embodiment of the described technology includes, but not limited thereto, a system, method, and computer readable medium that provides mobile device functional tremor retrainment technology, which illustrates a block diagram of an example machine 400 upon which one or more embodiments (e.g., discussed methodologies) can be implemented (e.g., run). For instance, example machine 400 may comprise an implementation of a mobile device, such as mobile devices 103, 202, or 203.

Examples of machine 400 can include logic, one or more components, circuits (e.g., modules), or mechanisms. Circuits are tangible entities configured to perform certain operations. In an example, circuits can be arranged (e.g., internally or with respect to external entities such as other circuits) in a specified manner. In an example, one or more computer systems (e.g., a standalone, client or server computer system) or one or more hardware processors (processors) can be configured by software (e.g., instructions, an application portion, or an application) as a circuit that operates to perform certain operations as described herein. In an example, the software can reside (1) on a non-transitory computer readable medium or (2) in a transmission signal. In an example, the software, when executed by the underlying hardware of the circuit, causes the circuit to perform the certain operations.

In an example, a circuit can be implemented mechanically or electronically. For example, a circuit can comprise dedicated circuitry or logic that is specifically configured to perform one or more techniques such as discussed above, such as including a special-purpose processor, a field programmable gate array (FPGA) or an application-specific integrated circuit (ASIC). In an example, a circuit can comprise programmable logic (e.g., circuitry, as encompassed within a general-purpose processor or other programmable processor) that can be temporarily configured (e.g., by software) to perform the certain operations. It will be appreciated that the decision to implement a circuit mechanically (e.g., in dedicated and permanently configured circuitry), or in temporarily configured circuitry (e.g., configured by software) can be driven by cost and time considerations.

Accordingly, the term “circuit” is understood to encompass a tangible entity, be that an entity that is physically constructed, permanently configured (e.g., hardwired), or temporarily (e.g., transitorily) configured (e.g., programmed) to operate in a specified manner or to perform specified operations. In an example, given a plurality of temporarily configured circuits, each of the circuits need not be configured or instantiated at any one instance in time. For example, where the circuits comprise a general-purpose processor configured via software, the general-purpose processor can be configured as respective different circuits at different times. Software can accordingly configure a processor, for example, to constitute a particular circuit at one instance of time and to constitute a different circuit at a different instance of time.

In an example, circuits can provide information to, and receive information from, other circuits. In this example, the circuits can be regarded as being communicatively coupled to one or more other circuits. Where multiple of such circuits exist contemporaneously, communications can be achieved through signal transmission (e.g., over appropriate circuits and buses) that connect the circuits. In embodiments in which multiple circuits are configured or instantiated at different times, communications between such circuits can be achieved, for example, through the storage and retrieval of information in memory structures to which the multiple circuits have access. For example, one circuit can perform an operation and store the output of that operation in a memory device to which it is communicatively coupled. A further circuit can then, at a later time, access the memory device to retrieve and process the stored output. In an example, circuits can be configured to initiate or receive communications with input or output devices and can operate on a resource (e.g., a collection of information).

The various operations of method examples described herein can be performed, at least partially, by one or more processors that are temporarily configured (e.g., by software) or permanently configured to perform the relevant operations. Whether temporarily or permanently configured, such processors can constitute processor-implemented circuits that operate to perform one or more operations or functions. In an example, the circuits referred to herein can comprise processor-implemented circuits.

Similarly, the methods described herein can be at least partially processor-implemented. For example, at least some of the operations of a method can be performed by one or processors or processor-implemented circuits. The performance of certain of the operations can be distributed among the one or more processors, not only residing within a single machine, but deployed across a number of machines. In an example, the processor or processors can be located in a single location (e.g., within a home environment, an office environment or as a server farm), while in other examples the processors can be distributed across a number of locations. The one or more processors can also operate to support performance of the relevant operations in a “cloud computing” environment or as a “software as a service” (SaaS). For example, at least some of the operations can be performed by a group of computers (as examples of machines including processors), with these operations being accessible via a network (e.g., the Internet) and via one or more appropriate interfaces (e.g., Application Program Interfaces (APIs).)

Example embodiments (e.g., apparatus, systems, or methods) can be implemented in digital electronic circuitry, in computer hardware, in firmware, in software, or in any combination thereof. Example embodiments can be implemented using a computer program product (e.g., a computer program, tangibly embodied in an information carrier or in a computer readable medium, for execution by, or to control the operation of, data processing apparatus such as a programmable processor, a computer, or multiple computers).

A computer program can be written in any form of programming language, including compiled or interpreted languages, and it can be deployed in any form, including as a standalone program or as a software module, subroutine, or other unit suitable for use in a computing environment. A computer program can be deployed to be executed on one computer or on multiple computers at one site or distributed across multiple sites and interconnected by a communication network.

In an example, operations can be performed by one or more programmable processors executing a computer program to perform functions by operating on input data and generating output. Examples of method operations can also be performed by, and example apparatus can be implemented as, special purpose logic circuitry (e.g., a field programmable gate array (FPGA) or an application-specific integrated circuit (ASIC)).

The computing system can include clients and servers. A client and server are generally remote from each other and generally interact through a communication network. The relationship of client and server arises by virtue of computer programs running on the respective computers and having a client-server relationship to each other. In embodiments deploying a programmable computing system, it will be appreciated that both hardware and software architectures require consideration. Specifically, it will be appreciated that the choice of whether to implement certain functionality in permanently configured hardware (e.g., an ASIC), in temporarily configured hardware (e.g., a combination of software and a programmable processor), or a combination of permanently and temporarily configured hardware can be a design choice. Below are set out hardware (e.g., machine 400) and software architectures that can be deployed in example embodiments. In an example, the machine 400 can operate as a standalone device or the machine 400 can be connected (e.g., networked) to other machines.

In a networked deployment, the machine 400 can operate in the capacity of either a server or a client machine in server-client network environments. In an example, machine 400 can act as a peer machine in peer-to-peer (or other distributed) network environments. The machine 400 can be a personal computer (PC), a tablet PC, a set-top box (STB), a Personal Digital Assistant (PDA), a mobile telephone, a web appliance, a network router, switch or bridge, or any machine capable of executing instructions (sequential or otherwise) specifying actions to be taken (e.g., performed) by the machine 400. Further, while only a single machine 400 is illustrated, the term “machine” shall also be taken to include any collection of machines that individually or jointly execute a set (or multiple sets) of instructions to perform any one or more of the methodologies discussed herein.

Example machine (e.g., computer system) 400 can include a processor 402 (e.g., a central processing unit (CPU), a graphics processing unit (GPU) or both), a main memory 404 and a static memory 406, some or all of which can communicate with each other via a bus 408. The machine 400 can further include a display unit 410, an alphanumeric input device 412 (e.g., a keyboard), and a user interface (UI) navigation device 411 (e.g., a mouse). In an example, the display unit 810, input device 417 and UI navigation device 414 can be a touch screen display. The machine 400 can additionally include a storage device (e.g., drive unit) 416, a signal generation device 418 (e.g., a speaker), a network interface device 420, and one or more sensors 421, such as a global positioning system (GPS) sensor, compass, accelerometer, or other sensor.

The storage device 416 can include a computer readable medium 422 on which is stored one or more sets of data structures or instructions 424 (e.g., software) embodying or utilized by any one or more of the methodologies or functions described herein. The instructions 424 can also reside, completely or at least partially, within the main memory 404, within static memory 406, or within the processor 402 during execution thereof by the machine 400. In an example, one or any combination of the processor 402, the main memory 404, the static memory 406, or the storage device 416 can constitute computer readable media.

While the computer readable medium 422 is illustrated as a single medium, the term “computer readable medium” can include a single medium or multiple media (e.g., a centralized or distributed database, and/or associated caches and servers) that configured to store the one or more instructions 424. The term “computer readable medium” can also be taken to include any tangible medium that is capable of storing, encoding, or carrying instructions for execution by the machine and that cause the machine to perform any one or more of the methodologies of the present disclosure or that is capable of storing, encoding, or carrying data structures utilized by or associated with such instructions. The term “computer readable medium” can accordingly be taken to include, but not be limited to, solid-state memories, and optical and magnetic media. Specific examples of computer readable media can include non-volatile memory, including, by way of example, semiconductor memory devices (e.g., Electrically Programmable Read-Only Memory (EPROM), Electrically Erasable Programmable Read-Only Memory (EEPROM)) and flash memory devices; magnetic disks such as internal hard disks and removable disks; magneto-optical disks; and CD-ROM and DVD-ROM disks.

The instructions 424 can further be transmitted or received over a communications network 426 using a transmission medium via the network interface device 420 utilizing any one of a number of transfer protocols (e.g., frame relay, IP, TCP, UDP, HTTP, etc.). Example communication networks can include a local area network (LAN), a wide area network (WAN), a packet data network (e.g., the Internet), mobile telephone networks (e.g., cellular networks), Plain Old Telephone (POTS) networks, and wireless data networks (e.g., IEEE 802.11 standards family known as Wi-Fi®, IEEE 802.16 standards family known as WiMax®), peer-to-peer (P2P) networks, among others. The term “transmission medium” shall be taken to include any intangible medium that is capable of storing, encoding, or carrying instructions for execution by the machine, and includes digital or analog communications signals or other intangible medium to facilitate communication of such software.

Although example embodiments of the present disclosure are explained in some instances in detail herein, it is to be understood that other embodiments are contemplated. Accordingly, it is not intended that the present disclosure be limited in its scope to the details of construction and arrangement of components set forth in the following description or illustrated in the drawings. The present disclosure is capable of other embodiments and of being practiced or carried out in various ways.

It should be appreciated that any element, part, section, subsection, or component described with reference to any specific embodiment above may be incorporated with, integrated into, or otherwise adapted for use with any other embodiment described herein unless specifically noted otherwise or if it should render the embodiment device nonfunctional. Likewise, any step described with reference to a particular method or process may be integrated, incorporated, or otherwise combined with other methods or processes described herein unless specifically stated otherwise or if it should render the embodiment method nonfunctional. Furthermore, multiple embodiment devices or embodiment methods may be combined, incorporated, or otherwise integrated into one another to construct or develop further embodiments of the invention described herein.

It should be appreciated that any of the components or modules referred to with regards to any of the present invention embodiments discussed herein, may be integrally or separately formed with one another. Further, redundant functions or structures of the components or modules may be implemented. Moreover, the various components may be communicated locally and/or remotely with any user/operator/customer/client or machine/system/computer/processor. Moreover, the various components may be in communication via wireless and/or hardwire or other desirable and available communication means, systems, and hardware. Moreover, various components and modules may be substituted with other modules or components that provide similar functions.

It should be appreciated that the device and related components discussed herein may take on all shapes along the entire continual geometric spectrum of manipulation of x, y and z planes to provide and meet the environmental, anatomical, and structural demands and operational requirements. Moreover, locations and alignments of the various components may vary as desired or required.

It should be appreciated that various sizes, dimensions, contours, rigidity, shapes, flexibility and materials of any of the components or portions of components in the various embodiments discussed throughout may be varied and utilized as desired or required.

It should be appreciated that while some dimensions are provided on the aforementioned figures, the device may constitute various sizes, dimensions, contours, rigidity, shapes, flexibility and materials as it pertains to the components or portions of components of the device, and therefore may be varied and utilized as desired or required.

It must also be noted that, as used in the specification and the appended claims, the singular forms “a,” “an” and “the” include plural referents unless the context clearly dictates otherwise. Ranges may be expressed herein as from “about” or “approximately” one particular value and/or to “about” or “approximately” another particular value. When such a range is expressed, other exemplary embodiments include from the one particular value and/or to the other particular value.

By “comprising” or “containing” or “including” is meant that at least the named compound, element, particle, or method step is present in the composition or article or method, but does not exclude the presence of other compounds, materials, particles, or method steps, even if the other such compounds, material, particles, or method steps have the same function as what is named.

In describing example embodiments, terminology will be resorted to for the sake of clarity. It is intended that each term contemplates its broadest meaning as understood by those skilled in the art and includes all technical equivalents that operate in a similar manner to accomplish a similar purpose. It is also to be understood that the mention of one or more steps of a method does not preclude the presence of additional method steps or intervening method steps between those steps expressly identified. Steps of a method may be performed in a different order than those described herein without departing from the scope of the present disclosure. Similarly, it is also to be understood that the mention of one or more components in a device or system does not preclude the presence of additional components or intervening components between those components expressly identified.

Some references, which may include various patents, patent applications, and publications, are cited in a reference list and discussed in the disclosure provided herein. The citation and/or discussion of such references is provided merely to clarify the description of the present disclosure and is not an admission that any such reference is “prior art” to any aspects of the present disclosure described herein. In terms of notation, “[n]” corresponds to the n^{th} reference in the list. All references cited and discussed in this specification are incorporated herein by reference in their entireties and to the same extent as if each reference was individually incorporated by reference.

It should be appreciated that as discussed herein, a subject may be a human or any animal. It should be appreciated that an animal may be a variety of any applicable type, including, but not limited thereto, mammal, veterinarian animal, livestock animal or pet type animal, etc. As an example, the animal may be a laboratory animal specifically selected to have certain characteristics similar to human (e.g., rat, dog, pig, monkey), etc. It should be appreciated that the subject may be any applicable human patient, for example.

As discussed herein, a “subject” may be any applicable human, animal, or other organism, living or dead, or other biological or molecular structure or chemical environment, and may relate to particular components of the subject, for instance specific tissues or fluids of a subject (e.g., human tissue in a particular area of the body of a living subject), which may be in a particular location of the subject, referred to herein as an “area of interest” or a “region of interest.”

The term “about,” as used herein, means approximately, in the region of, roughly, or around. When the term “about” is used in conjunction with a numerical range, it modifies that range by extending the boundaries above and below the numerical values set forth. In general, the term “about” is used herein to modify a numerical value above and below the stated value by a variance of 10%. In one aspect, the term “about” means plus or minus 10% of the numerical value of the number with which it is being used. Therefore, about 50% means in the range of 45%-55%. Numerical ranges recited herein by endpoints include all numbers and fractions subsumed within that range (e.g., 1 to 5 includes 1, 1.5, 2, 2.75, 3, 3.90, 4, 4.24, and 5). Similarly, numerical ranges recited herein by endpoints include subranges subsumed within that range (e.g., 1 to 5 includes 1-1.5, 1.5-2, 2-2.75, 2.75-3, 3-3.90, 3.90-4, 4-4.24, 4.24-5, 2-5, 3-5, 1-4, and 2-4). It is also to be understood that all numbers and fractions thereof are presumed to be modified by the term “about.”

As used herein, unless context dictates otherwise, the terms “quick” and “slow” and inflections refer to frequency, not speed. For instance, description of a user moving “too slowly” refers generally to the user moving at a frequency that is lower than a threshold, not to the user moving their hand through space at too slow a speed.

In summary, while the present technology has been described with respect to specific embodiments, many modifications, variations, alterations, substitutions, and equivalents will be apparent to those skilled in the art. The present invention is not to be limited in scope by the specific embodiment described herein. Indeed, various modifications of the present invention, in addition to those described herein, will be apparent to those of skill in the art from the foregoing description and accompanying drawings. Accordingly, the invention is to be considered as limited only by the spirit and scope of the disclosure (and claims) including all modifications and equivalents.

Still other embodiments will become readily apparent to those skilled in the art from reading the above-recited detailed description and drawings of certain exemplary embodiments. It should be understood that numerous variations, modifications, and additional embodiments are possible, and accordingly, all such variations, modifications, and embodiments are to be regarded as being within the spirit and scope of this application. For example, regardless of the content of any portion (e.g., title, field, background, summary, abstract, drawing figure, etc.) of this application, unless clearly specified to the contrary, there is no requirement for the inclusion in any claim herein or of any application claiming priority hereto of any particular described or illustrated activity or element, any particular sequence of such activities, or any particular interrelationships of such elements. Moreover, any activity can be repeated, any activity can be performed by multiple entities, or any element can be duplicated. Further, any activity or element can be excluded, the sequence of activities can vary, and/or the interrelationships of elements can vary. Unless clearly specified to the contrary, there is no requirement for any particular described or illustrated activity or element, any particular sequence or such activities, any particular size, speed, material, dimension or frequency, or any particular interrelationship of such elements. Accordingly, the descriptions and drawings are to be regarded as illustrative in nature, and not as restrictive. Moreover, when any number or range is described herein, unless clearly stated otherwise, that number or range is approximate. Wen any range is described herein, unless clearly stated otherwise, that range includes all values therein and all sub ranges therein. Any information in any material (e.g., a United States/foreign patent, United States/foreign patent application, book, article, etc.) that has been incorporated by reference herein, is only incorporated by reference to the extent that no conflict exists between such information and the other statements and drawings set forth herein. In the event of such conflict, including a conflict that would render invalid any claim herein or seeking priority hereto, then any such conflicting information in such incorporated by reference material is specifically not incorporated by reference herein.

What is claimed is:

1. A mobile device, comprising:
a processor;
a user interface; and
a memory storing instructions to cause the processor to:
measure a baseline movement frequency of a limb of a user, using motion data from a sensor mounted on the limb;
determine a cue frequency based on the baseline movement frequency;
provide motion cues to the user, via the user interface, during a training session, wherein the motion cues exhibit the cue frequency for the user to follow;
measure a training session motion frequency of the limb using motion data from the sensor while providing the motion cues; and
provide a feedback signal to the user via the user interface, the feedback signal indicative of a synchrony of the training session motion frequency to the cue frequency.

2. The mobile device of claim 1, wherein the mobile device comprises a display used to provide the feedback signal, a speaker used to provide the motion cues, and an accelerometer to sense the motion data.

3. The mobile device of claim 1, wherein the instructions are to cause the processor to:
receive the motion data from a second mobile device;
provide the motion cues via the second mobile device; and
provide the feedback signal via the second mobile device;
wherein the second mobile device comprises an accelerometer, a display, and a speaker.

4. The mobile device of claim 1, wherein the instructions are to cause the processor to provide the feedback signal continuously while providing the motion cues.

5. The mobile device of claim 1, wherein:
the feedback signal comprises a positive feedback signal when the training session motion frequency is within a range of the cue frequency; and
the feedback signal comprises a negative feedback signal when the training session motion frequency is outside the range of the cue frequency.

6. The mobile device of claim 1, wherein the instructions are to cause the processor to:
receive input indicative of a treatment session duration from a healthcare provider; and
determine a treatment session length based on the input.

7. The mobile device of claim 1, wherein the instructions are to cause the processor to:
determine a first cue frequency based on the baseline movement frequency and a second cue frequency based on the baseline movement frequency;
provide motion cues to the user at the first cue frequency for a first portion of a session; and
provide motion cues to the user at the second cue frequency for a second portion of the session.

8. A non-transitory computer readable medium storing instructions executable by a processor to cause a mobile device to:
measure a baseline tremor frequency of an extended arm of a user, via accelerometer data obtained from an accelerometer mounted on a wrist of the extended arm;
determine a cue frequency based on the baseline tremor frequency;
provide motion cues at the cue frequency to the user;
measure a motion frequency of the extended arm using the accelerometer while providing the motion cues; and
provide a feedback signal indicative of a synchrony of the motion frequency to the cue frequency.

9. The non-transitory computer readable medium of claim 8, storing instructions executable by the processor to cause the mobile device to:
determine a first cue frequency based on the baseline tremor frequency and a second cue frequency based on the baseline tremor frequency;
provide motion cues to the user at the first cue frequency for a first portion of a session; and
provide motion cues to the user at the second cue frequency for a second portion of the session.

10. The non-transitory computer readable medium of claim 8, storing instructions executable by the processor to cause the mobile device to:
receive user input indicative of a treatment session; and
determine a treatment session length based on the input.

11. The non-transitory computer readable medium of claim 8, storing instructions executable by the processor to cause the mobile device to provide the feedback signal continuously while providing the motion cues.

12. The non-transitory computer readable medium of claim 8, wherein:
the feedback signal comprises a positive feedback signal when the motion frequency is within a range of the cue frequency; and
the feedback signal comprises a negative feedback signal when the motion frequency is outside the range of the cue frequency.

13. The non-transitory computer readable medium of claim 8, wherein the mobile device comprises the processor, the accelerometer, a speaker to provide the motion cues, and a display to provide the feedback signal.

14. A method, comprising:
measuring, via a mobile device monitoring a patient, an initial tremor frequency of a portion of the patient's body exhibiting functional movement disorder;
determining a cue frequency based on the initial tremor frequency, the cue frequency being different than the initial tremor frequency;
providing motion cues to the patient at the cue frequency, via the mobile device;
measuring a motion frequency of the portion of the patient's body while providing the motion cues; and
providing a feedback signal to the patient via the mobile device, indicative of a degree of synchrony of the motion frequency to the cue frequency.

15. The method of claim 14, wherein the feedback signal comprises a visual feedback signal displayed on a screen of the mobile device while providing the motion cues.

16. The method of claim 15, wherein the feedback signal comprises a gauge graphic providing a visual indication of a difference between the motion frequency and the cue frequency.

17. The method of claim 14, further comprising:
the mobile device receiving input indicative of a treatment session setting from a healthcare provider of the patient; and
the mobile device determining an attribute of deployment of the treatment session based on the input, the attribute comprising at least one of: treatment session duration, cue frequency, and a format of the feedback signal.

18. The method of claim 14, further comprising:
the mobile device determining a first cue frequency based on the initial tremor frequency and a second cue frequency based on the initial tremor frequency;
the mobile device providing motion cues to the patient at the first cue frequency for a first portion of a session; and
the mobile device providing motion cues to the user at the second cue frequency for a second portion of the session.

19. The method of claim 18, wherein the first cue frequency is greater than 50% of the baseline tremor frequency and the second cue frequency is less than 50% of the baseline tremor frequency.

20. The method of claim 14, wherein:
the feedback signal comprises a positive feedback signal when the motion frequency is within a range of the cue frequency; and
the feedback signal comprises a negative feedback signal when the motion frequency is outside the range of the cue frequency.
